(19) United States
(12) Patent Application Publication
(10) Pub. No.: US 2022/0412986 A1
(43) Pub. Date: Dec. 29, 2022

(54) NORMALISATION METHOD AND NORMALISATION SYSTEM FOR A DRIED BLOOD MATRIX

(71) Applicant: Labor Berlin - Charité Vivantes Services GmbH, Berlin (DE)

(72) Inventors: Tina BUCHHOLZ, Berlin (DE); Nancy KRÜGER, Brandenburg (DE); Oliver BLANKENSTEIN, Berlin (DE); Falko BÖHRINGER, Berlin (DE)

(73) Assignee: Labor Berlin - Charité Vivantes Services GmbH, Berlin (DE)

(21) Appl. No.: 17/780,980

(22) PCT Filed: Nov. 27, 2020
(86) PCT No.: PCT/EP2020/083627

§ 371 Date: May 29, 2022

(30) Foreign Application Priority Data
Nov. 29, 2019 (DE) 10 2019 218 593.2

(51) Int. Cl. G01N 33/68; G01N 33/72

(52) U.S. Cl. CPC G01N 33/6848; G01N 33/721

(57) ABSTRACT

The present invention relates to a normalisation method for a dried blood matrix, said method comprising the steps: providing the dried blood matrix, extracting at least one analyte from a first portion of the dried blood matrix and haemoglobin from a second portion of the dried blood matrix, quantitatively analysing the at least one extracted analyte and the extracted haemoglobin, determining a concentration of the at least one analyte in the first portion of the dried blood matrix and a concentration of the haemoglobin in the second portion of the dried blood matrix, deriving the haematocrit of the second portion of the dried blood matrix on the basis of the concentration of the haemoglobin, and calculating a normalisation factor on the basis of the determined haematocrit in order to normalise the concentration of the at least one analyte in the first portion of the dried blood matrix. The invention also relates to a normalisation device (10) for performing a method according to the invention.

The present invention relates to a normalisation method and a normalisation system for a dried blood matrix for the normalised determination of at least one analyte in the dried blood matrix.

The routine analysis of clinically relevant parameters has in the past usually been carried out on liquid blood sample matrices in the form of whole blood, serum or plasma. The reference ranges or standard concentrations for almost all clinical parameters are, accordingly, defined for liquid sample matrices. Since all conditions and components that control and ensure the degradation of analytes in vivo are still present in the liquid sample matrices, complex measures, for example in the form of enzyme blockade and/or cooling, often have to be taken in order to prevent these processes and thus determine the correct concentrations of the analyte in the laboratory. Most degradation processes are time-critical and temperature-dependent. The only exceptions are metabolic end products that do not undergo further enzymatic degradation.

Newborn screening occupies a special position in routine high-throughput analysis, as dried blood is used as a sample matrix for the analysis. Dried blood has to be used for this analysis in particular because liquid blood is only available in very limited quantities in newborns. The analysis of dried blood in newborn screening is currently carried out in combination with highly sensitive special analysis methods.

In order to generate the required dried blood, whole blood is applied to special, absorbent carrier materials. The liquid contained in the blood is absorbed into the carrier material due to the chromatography effect, as a result of which the blood is dried within a short time. The drying prevents enzymatic degradation processes. Due to the partially planar dimension of the dried blood carrier material, the dried blood samples can be transported in a simple and inexpensive way by post. In addition, dried blood is not infectious and does not require a special declaration.

Despite the advantages mentioned, dried blood has not yet found widespread use in clinical routine analysis. This is because all dried blood analyses based on whole blood as a sample matrix are subject to fluctuations that make it difficult to impossible to achieve meaningful analysis results. In addition, a comparability with the established analyses from liquid sample matrices is not possible.

The object of the present invention is to take into account at least partially the problems described above. In particular, it is object of the present invention to create an improved normalisation method and an improved normalisation system for a dried blood matrix.

The above object is achieved by the claims. In particular, the above object is achieved by the normalisation method according to claim 1 and the normalisation system according to claim 9. Further advantages of the invention emerge from the dependent claims, the description and the drawings. Features and details which are described in connection with the normalisation method naturally also apply in connection with the normalisation system according to the invention and vice versa, so that with regard to disclosure, mutual reference is or can always made to the individual aspects of invention.

According to a first aspect of the present invention, a normalisation method for a dried blood matrix is provided. The normalisation method comprises the following steps:
providing the dried blood matrix,
extracting at least one analyte from a first portion of the dried blood matrix and haemoglobin from a second portion of the dried blood matrix,
quantitatively analysing the at least one extracted analyte and the extracted haemoglobin,
determining a concentration of the at least one analyte in the first portion of the dried blood matrix and a concentration of the haemoglobin in the second portion of the dried blood matrix,
deriving a haematocrit of the second portion of the dried blood matrix on the basis of the concentration of the haemoglobin,
calculating a normalisation factor on the basis of the determined haematocrit in order to normalise the concentration of the at least one analyte in the first portion of the dried blood matrix.

The normalisation method according to the invention is universally applicable to a variety of clinically relevant analytes. The method can also be applied to species other than humans, in particular to all vertebrates. With the help of the invention, a more detailed analysis carried out on dried blood as a sample matrix can be made more widely available to the laboratory market. Due to the flexible and basically non-time-critical handling of dried blood matrices, previously incurred logistics costs can be greatly reduced.

The first portion of the dried blood matrix and/or the second portion of the dried blood matrix can in each case be provided in the form of a dried blood spot by manual or automatic punching. That is to say, at least one first dried blood spot for the analyte and a second dried blood spot for the haemoglobin to be analysed can be obtained, in particular punched out, from the dried blood matrix. The at least one first dried blood spot and the second dried blood spot are then preferably prepared separately.

According to the invention, a plurality of analytes can be extracted from the at least one first dried blood spot or from the first portion of the dried blood matrix, or in each case one analyte can be extracted from a plurality of dried blood spots or corresponding portions of the dried blood matrix.

The haemoglobin is preferably extracted using an aqueous buffer. Quantitative analysis is preferably to be understood in the present case as a quantitative determination and/or a corresponding measurement. That is to say, the quantity of the respective substance is determined through the quantitative analysis of the at least one extracted analyte and the extracted haemoglobin. The quantitative analysis of the haemoglobin is preferably carried out by mass spectrometry, by means of enzymatic fragmentation. Extensive experiments conducted in connection with the present invention have shown that an enzymatic cleavage of the haemoglobin is not necessarily required and in some partial aspects could lead to disadvantages during extraction. That is to say, with an advantageous alternative extraction, an enzymatic cleavage of the haemoglobin can be dispensed with.

The quantitative analysis of the analyte and of the haemoglobin can be carried out simultaneously or separately. The highly sensitive analysis is preferably carried out as part of an analysis using liquid chromatography electrospray mass spectrometry. With a mass spectrometric measurement, haemoglobin and/or enzymatic fragments of the haemoglobin can also be detected by means of an MRM (Multiple Reaction Monitoring) process.

By determining the concentration of the at least one analyte in the first portion of the dried blood matrix and the concentration of the haemoglobin in the second portion of the dried blood matrix, the content of the substance is determined from the respective portion of the dried blood matrix. The haematocrit of the second portion of the dried blood matrix can then be derived based on the concentration of the haemoglobin. The derivation from the haemoglobin concentration preferably takes place via a linear relationship between the haemoglobin concentration and the haematocrit. In order to determine the concentration of the at least one analyte and of the haemoglobin, a calibration function can be used through which an area of the signals can be translated into a concentration. The derivation of the haematocrit can be performed on the basis of a translation of the haemoglobin concentration into haematocrit.

The haematocrit can be calculated according to the relationship

$$\frac{cHb \cdot [ \frac{g}{dL} ]}{3} = HKT$$

where cHb stands for the haemoglobin concentration.

Extracting haemoglobin can be understood to mean extracting haemoglobin and/or haemoglobin fragments. Accordingly, determining the concentration of the haemoglobin can be understood to mean determining the concentration of the haemoglobin and/or determining the concentration of the haemoglobin fragments. Accordingly, in this case haemoglobin can also be understood to refer to haemoglobin fragments.

The normalisation factor can be calculated and/or determined on the basis of various experimental series through which an adapted mathematical relationship can be arrived at empirically.

According to a further embodiment of the present invention it is possible that, for quantitative analysis of the haemoglobin, at least one peptide fragment of the haemoglobin is quantitatively analysed and then a concentration of the at least one peptide fragment in the second portion of the dried blood matrix is determined, wherein the concentration of the haemoglobin in the second portion of the dried blood matrix can be concluded on the basis of the concentration of the at least one peptide fragment. This is in particular advantageous in the simultaneous analysis of a plurality of analytes. That is to say, if a plurality of analytes are extracted from the dried blood matrix simultaneously, specific peptide fragments of the haemoglobin can be quantitatively determined, preferably simultaneously, during the quantitative analysis of the haemoglobin.

It is also possible that in a normalisation method according to the present invention the quantitative analysis of the haemoglobin or of the at least one peptide fragment is carried out on the basis of a repeated selection of analyte-specific mass fragments generated in a mass spectrometer. This can be carried out in an advantageous manner using a quadrupole mass spectrometer as part of an MRM process. It has proven to be particularly efficient if the quantitative analysis of the haemoglobin or of the at least one peptide fragment is carried out on the basis of a two-times selection of analyte-specific mass fragments generated in a mass spectrometer.

Furthermore, it is possible that the quantitative analysis of the haemoglobin or of the at least one peptide fragment is carried out in a normalisation method according to the invention on the basis of analyte-specific mass-to-charge ratio signals, m/z. Here, the haemoglobin and/or the at least one peptide fragment is, advantageously, selected on the basis of a mass spectrometry, not according to the mass alone, but on the basis of a mass/charge ratio. This allows particularly accurate analysis results to be achieved.

It can be of further advantage if, in a normalisation method according to the invention, for the quantitative analysis of the haemoglobin the haemoglobin is enzymatically cleaved through the addition of a peptidase. This allows meaningful analysis values to be generated which form an advantageous basis for further development with regard to the desired normalisation factor.

According to a further embodiment of the present invention it is possible that in a normalisation method a plasma equivalent concentration of the at least one analyte is determined on the basis of the normalisation factor. The plasma equivalent concentration of the at least one analyte is to be understood as a concentration which is analogous to a conventional plasma and/or serum concentration of an analyte from a plasma and/or serum sample. That is to say, the determined normalisation factor can be used to normalise the analyte concentration in the dried blood sample and to calculate a concentration analogous to the previously established plasma and/or serum concentration, i.e. a plasma equivalent concentration. The corresponding haematocrit normalisation of the analyte concentration means that quantitative analyses from dried blood matrices and from liquid sample matrices are directly comparable with each other.

A further advantage of the present invention arises if, in a normalisation method, the at least one analyte is extracted from the dried blood matrix by means of an extraction liquid. This allows the analyte to be extracted from the dried blood matrix in a simple and reliable way. Preferably, the at least one analyte is extracted from the dried blood matrix by means of rehydration. Furthermore, it has proved advantageous if the at least one analyte is extracted from the dried blood matrix using an organic or partially organic liquid, without water content or with a low water content of, for example, less than 10% by weight. Furthermore, in experiments conducted in connection with the present invention, it has also transpired, surprisingly, that the at least one analyte can also, advantageously, be extracted from the dried blood matrix by means of electromagnetic radiation. The at least one extracted analyte can then be converted into a gas phase for further use, in particular for the subsequent quantitative analysis.

According to a further aspect of the present invention, a normalisation system for a dried blood matrix is provided. The normalisation system comprises the following components:
an extraction unit for extracting at least one analyte from a first portion of the dried blood matrix and haemoglobin from a second portion of the dried blood matrix,
an analysis unit for quantitatively analysing the at least one extracted analyte and the extracted haemoglobin,
a determination unit for determining a concentration of the at least one analyte in the first portion of the dried blood matrix and a concentration of the haemoglobin in the second portion of the dried blood matrix,
a derivation unit for deriving a haematocrit of the second portion of the dried blood matrix on the basis of the concentration of the haemoglobin, and
a calculation unit for calculating a normalisation factor on the basis of the determined haematocrit in order to normalise a concentration of the at least one analyte in the first portion of the dried blood matrix.

Thus, a normalisation system according to the invention brings the same advantages as those described in detail with regard to the normalisation method according to the invention. The normalisation system may also be configured for calculating a normalisation factor according to a normalisation method described in detail above.

Further measures to improve the invention are disclosed in the following description of various exemplary embodiments of the invention, which are represented schematically in the figures. All features and/or advantages resulting from the claims, the description or the drawing, including constructive details and spatial arrangements, can be essential to the invention both in themselves and in the various combinations.

In each case schematically:

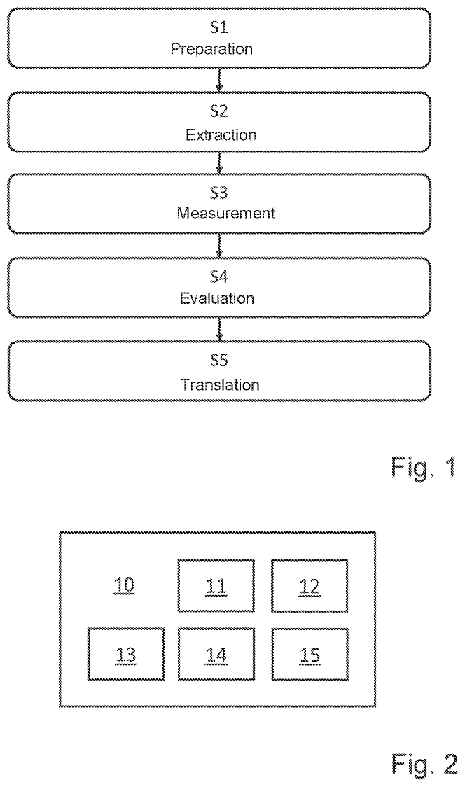

FIG. 1 shows a flowchart explaining a preferred embodiment of the present invention, and

FIG. 2 shows a block diagram representing a normalisation system according to the invention.

FIG. 1 shows a block diagram explaining a normalisation method for a dried blood matrix. According to the embodiment shown, in a preparation step S1 a dried blood matrix is first provided, from which a first dried blood spot for the extraction of the analyte and a second dried blood spot for the extraction of haemoglobin are punched out.

In a second step S2, the analyte is extracted from the first dried blood spot by means of organic solvent and the haemoglobin is extracted from the second dried blood spot by means of an aqueous buffer.

In the context of measurements carried out in a third step S3, the extracted analyte and the extracted haemoglobin are then quantitatively analysed on the basis of a two-times selection of analyte-specific mass fragments generated in a mass spectrometer. That is to say, in each case an absolute amount of the respective substance is determined. For quantitative analysis of the haemoglobin, peptide fragments of the haemoglobin are quantitatively analysed and a concentration of the peptide fragments in the second dried blood spot is then determined, wherein the concentration of the haemoglobin in the second dried blood spot is concluded on the basis of the concentration of the peptide fragments. In addition, for the quantitative analysis of the haemoglobin the haemoglobin is enzymatically cleaved through the addition of a peptidase.

In a further step S4, the measured data or the analysis results are evaluated. In particular, a concentration of the at least one analyte in the first dried blood spot and a concentration of the haemoglobin in the second dried blood spot are determined.

In a subsequent fifth step S5, on the basis of the evaluation results the haematocrit of the second dried blood spots is derived on the basis of the determined haemoglobin concentration and a normalisation factor is calculated on the basis of the haematocrit in order to normalise the concentration of the analyte. During the translation, a plasma equivalent concentration of the at least one analyte is also determined on the basis of the normalisation factor.

FIG. 2 shows a normalisation system 10 for the dried blood matrix which is configured for carrying out a normalisation method as described above. The normalisation system 10 includes an extraction unit 11 for extracting at least one analyte from a first portion of the dried blood matrix or the first dried blood spot and haemoglobin from a second portion of the dried blood matrix or the second dried blood spot. The normalisation system also includes an analysis unit 12 for quantitatively analysing the at least one extracted analyte and the extracted haemoglobin and a determination unit 13 for determining a concentration of the at least one analyte in the first portion of the dried blood matrix and a concentration of the haemoglobin in the second portion of the dried blood matrix. In addition, the normalisation system includes a derivation unit 14 for deriving a haematocrit of the second portion of the dried blood matrix on the basis of the concentration of haemoglobin and a calculation unit 15 for calculating a normalisation factor on the basis of the determined haematocrit in order to normalise a concentration of the at least one analyte in the first portion of the dried blood matrix.

In addition to the embodiments illustrated, the invention allows for further design principles, i.e. the invention should not be considered to be limited to the embodiment illustrated in the Figures.

### LIST OF REFERENCE SIGNS

10 normalisation system
11 extraction unit
12 analysis unit
13 determination unit
14 derivation unit
15 calculation unit

1. Normalisation method for a dried blood matrix, comprising the steps:
providing the dried blood matrix,
extracting at least one analyte from a first portion of the dried blood matrix and haemoglobin from a second portion of the dried blood matrix,
quantitatively analysing the at least one extracted analyte and the extracted haemoglobin,
determining a concentration of the at least one analyte in the first portion of the dried blood matrix and a concentration of the haemoglobin in the second portion of the dried blood matrix,
deriving a haematocrit of the second portion of the dried blood matrix on the basis of the concentration of the haemoglobin, and
calculating a normalisation factor on the basis of the determined haematocrit in order to normalise the concentration of the at least one analyte in the first portion of the dried blood matrix.

2. Normalisation method according to claim 1,
characterised in that
for the quantitative analysis of the haemoglobin, at least one peptide fragment of the haemoglobin is quantitatively analysed and a concentration of the at least one peptide fragment in the second portion of the dried blood matrix is then determined, wherein the concentration of the haemoglobin in the second portion of the dried blood matrix is concluded on the basis of the concentration of the at least one peptide fragment.

3. Normalisation method according to claim 1,
characterised in that
the quantitative analysis of the haemoglobin or of the at least one peptide fragment is carried out on the basis of a repeated selection of analyte-specific mass fragments generated in a mass spectrometer.

4. Normalisation method according to claim 1,
characterised in that
the quantitative analysis of the haemoglobin or of the at least one peptide fragment is carried out on the basis of analyte-specific mass-to-charge ratio signals, m/z.

5. Normalisation method according to claim 1,
characterised in that
for the quantitative analysis of the haemoglobin, the haemoglobin is enzymatically cleaved through the addition of a peptidase.

6. Normalisation method according to claim 1,
characterised in that
a plasma equivalent concentration of the at least one analyte is determined on the basis of the normalisation factor.

7. Normalisation method according to claim 1,
characterised in that
the at least one analyte is extracted from the dried blood matrix by means of an extraction liquid.

8. Normalisation method according to claim 1,
characterised in that
the at least one analyte is extracted from the dried blood matrix using an organic solvent.

9. Normalisation system (10) for a dried blood matrix, comprising
an extraction unit (11) for extracting at least one analyte from a first portion of the dried blood matrix and haemoglobin from a second portion of the dried blood matrix,
an analysis unit (12) for quantitatively analysing the at least one extracted analyte and the extracted haemoglobin,
a determination unit (13) for determining a concentration of the at least one analyte in the first portion of the dried blood matrix and a concentration of the haemoglobin in the second portion of the dried blood matrix,
a derivation unit (14) for deriving a haematocrit of the second portion of the dried blood matrix on the basis of the concentration of the haemoglobin, and
a calculation unit (15) for calculating a normalisation factor on the basis of the determined haematocrit in order to normalise a concentration of the at least one analyte in the first portion of the dried blood matrix.

10. (canceled)
